(19) United States
(12) Patent Application Publication
(10) Pub. No.: US 2010/0041970 A1
(43) Pub. Date: Feb. 18, 2010

(54) IMPLANTABLE MEDICAL DEVICE

(75) Inventors: Sven-Erik Hedberg, Kungsangen (SE); Andreas Blomqvist, Spanga (SE); Karin Järverud, Solna (SE); Karin Ljungström, Hasselby (SE); Leif Lindkvist, Stenhamra (SE); Urban Lönn, Uppsala (SE); Nils Holmström, Jarfalla (SE)

Correspondence Address: SCHIFF HARDIN, LLP;PATENT DEPARTMENT, 233 S. Wacker Drive-Suite 6600, CHICAGO, IL, 60606-6473, US

(73) Assignee: St. Jude Medical AB Corproation

(21) Appl. No.: 12/532,186

(22) PCT Filed: Nov. 27, 2007
(86) PCT No.: PCT/SE07/01044

§ 371 Date: Sep. 21, 2009

(30) Foreign Application Priority Data
Mar. 23, 2007 (SE) PCT/SE2007/000289

(51) Int. Cl. A61B 5/1459

(52) U.S. Cl. 600/333

(57) ABSTRACT

An implantable medical device has an oxygen sensor adapted to measure the level of oxygen in oxygenized blood, and to generate an oxygen measurement signal in dependence of the level of oxygen. The oxygen sensor is adapted to perform measurements inside the heart, of blood entering the left atrium of a patient's heart. The obtained oxygen measurement signal is compared to a predetermined threshold level and an indication signal is generated in dependence of the comparison. The, indication signal is indicative of the lung functionality of the patient.

### BACKGROUND OF THE INVENTION

1. Field of the Invention

The present invention concerns an implantable medical device of the type having an optical oxygen sensor that measures the level of oxygen in oxygenated blood, and that generates an oxygen measurement signal dependent on the level of oxygen.

Description of the Prior Art

Insufficiency of the lungs, like in Chronic Obstructive Pulmonary Disease (COPD), pulmonary edema due to Heart Failure, asthma, chronic bronchitis, emphysema and interstitial lung diseases have high prevalence adding extensive costs to the health care system and suffering for the patients. Therefore, it is a general fact that enhanced management of these patients would be useful.

In the following a number of publications are briefly discussed providing relevant background information in the field of diagnostic methods and apparatuses for detecting insufficiencies of the lungs.

United States Patent Application Publication No. 2003/0149367 relates to a system and method for evaluating risk of mortality due to congestive heart failure using physiological sensors. The method includes that arterial oxygen saturation is measured. Electrodes may be place in the left atrium for atrial pacing. It is mentioned that a combined pacemaker lead and physiological sensor can be used.

EP-1102198 relates to an automated collection and analysis patient care system and method for diagnosing and monitoring respiratory insufficiency and outcomes thereof. The system includes an implantable device for measuring the arterial oxygen saturation.

EP-1151718 relates to an apparatus for monitoring heart failure via respiratory patterns. An implantable device is adapted to measure the arterial oxygen saturation in order to detect changes in the breathing pattern.

U.S. Pat. No. 7,010,337 discloses method and apparatus for monitoring blood condition and cardiopulmonary function, where sensors located on a sensor carrier are placed adjacent one or more of a surgical patient's major thoracic blood-containing structures such as the aorta or pulmonary artery, and characteristics of the blood in the blood-containing structures are determined non-invasively by measuring transmission or reflection of light or other types of energy by the blood.

Further improvements of today's technology are needed in order to meet the increasing demands of devices that enable accurate and reliable detection of lung insufficiencies.

### SUMMARY OF THE INVENTION

An object of the present invention is to provide a device that has improved capabilities regarding detection and monitoring of a patient's lung function, which may be related to Chronic Obstructive Pulmonary Disease (COPD), pulmonary oedema due to Heart Failure, asthma, chronic bronchitis, emphysema and interstitial lung diseases.

The above object is achieved in accordance with the present invention in an implantable medical device having an oxygen sensor that measures the level of oxygen in oxygenated blood in order to generate an oxygen measurement signal dependent on the level of oxygen, wherein the oxygen sensor performs the measurements in the heart, with regard to blood entering the left atrium of the heart, and wherein the obtained oxygen measurement signal is compared to a predetermined threshold level, and, as a result of the comparison, an indication signal is generated that indicates the lung functionality of the subject.

According to the present invention the degree of oxygen saturation of the blood oxygenized by the lungs, is monitored by an optical blood oxygen saturation sensor.

In particular the oxygen saturation measurement is performed when specified measurement criteria is fulfilled.

According to one embodiment the sensor is placed in or at the left atrium, which directly receives the oxygenized blood from the lungs. A preferred placement of the sensor is in the septum wall between the right and left atria.

An alternative placement is at the endocardium of the left atrium in order to minimise the risk of forming thrombosis.

According to another embodiment the oxygen sensor is mechanically integrated or co-assembled with a pressure sensor.

The sensor is connected to an implantable medical device (IMD). The device is provided with a measuring unit that measures the blood oxygen saturation detected by the oxygen sensor at suitable points in time. At a time, when the patient carrying this IMD, is at rest or asleep, the interference from other sources, that might disturb the sensor measurements, preferably is at a minimum. This is often the best choice of time to perform these measurements. The blood leaving the lungs has normally a high SaO₂ value even during high patient workload.

According to an embodiment of the present invention, the degree of oxygen saturation of the blood oxygenized by the lungs is monitored by means of photoluminescence sensor utilizing photoluminescent molecules emitting light as a response to the concentration of oxygen in blood.

In embodiments of the invention, the oxygen sensor is a pO₂ sensor, for example, a sensor as described in U.S. Pat. No. 5,431,172 sensitive to the levels of oxygen in arterial blood. It has been shown that the SvO₂ and SaO₂ values are correlated with the partial pressure of oxygen, pO₂, in blood and thus SaO₂ values may easily be calculated from measured pO₂ values, and vice versa.

In case a lung problem evolves, which causes inadequate oxygenation, this will be measured by the sensor. SaO₂ data will be stored and time marked in the IMD and analyzed. The stored data shall be used for diagnostic purposes and by the algorithm which will detect when the SaO₂ values are below a certain limit for more than a certain period of time and at conditions which are recognized to give reliable sensor data. Normal patient activity will be recognized by data from an auxiliary sensor, such as an activity sensor. The SaO₂ data should normally only be accepted when the activity sensor data indicate a patient at rest. In order to get reliable sensor data they shall be sampled at several times during this period of inactivity and the data shall be at about the same amplitude.

Disturbing influences, such as flying at a high altitude, may result in wrong interpretation of the status of the patient. Other auxiliary sensors, such as accelerometers and posture sensors, are then useful for being able to distinguish flying at high altitude from having impaired oxygenation in the lungs, e.g. by not performing blood oxygen saturation measurements if auxiliary sensors indicate that the patient is flying at a high altitude.

The present invention may also be used for detecting when a patient no longer may saturate the blood during exercise. This may be used for diagnostic purposes to show the physician at which work load this desaturation starts.

The usage of a SaO₂ sensor in the left atrium may also be used together with saturation sensors in other parts of the heart or body of the patient. In many cases those algorithms using blood saturation sensors anticipate full saturation of the blood leaving the lungs. An example of that would be in a pacemaker provided with a rate responsive function based on the oxygen saturation in the right ventricle.

The SaO₂ value may also be used for detecting impaired oxygenation in the lungs caused by pulmonary edema. The SaO₂ sensor may not differentiate pulmonary edema from other lung diseases, but using impedance measurements internally over parts of thorax may indicate that such a condition is at hand.

SaO₂-values may be transferred to e.g. databases and physicians using conventional telemetry communication and further on via internet on regular or planned occasions as well as when SaO₂ alarm limits have been passed.

### BRIEF DESCRIPTION OF THE DRAWINGS

FIG. 1 is a schematic block diagram illustrating the present invention.

FIG. 2 is a schematic block diagram illustrating one embodiment of the present invention.

FIG. 3 is a schematic block diagram illustrating another embodiment of the present invention.

FIG. 4 is a schematic illustration of an electrode lead according to the present invention, where the oxygen sensor is arranged; a sectional side view, and a view from the distal side of the lead (to the right in the figure).

FIG. 5 is a schematic illustration of an electrode lead according to one embodiment of the present invention, where the oxygen sensor is arranged; a sectional side view, and a view from the distal side of the lead (to the right in the figure).

FIG. 6 is a schematic illustration of an implantable medical device according to the invention provided with two electrode leads.

FIG. 7 is a schematic illustration of the p O₂ sensor according to one embodiment of the present invention.

FIG. 8 is a schematic illustration of the p O₂ sensor according to one embodiment of the present invention.

### DESCRIPTION OF THE PREFERRED EMBODIMENTS

The oxygen saturation of blood is generally defined as the percentage of the hemoglobin binding sites that are occupied with an oxygen molecule. An SaO₂ value in the range of 97-100% is considered normal, and an SaO₂ value in the range of 92-97% indicates interstitial edema and should trigger an alarm to avoid pulmonary edema and hospitalization (this is further discussed below). An SaO₂ value below 92% indicates pulmonary edema and the patient should immediately come to hospital.

FIG. 1 is a schematic block diagram illustrating the present invention. The implantable medical device according to the present invention comprises an optical oxygen sensor adapted to measure the level of oxygen in oxygenized blood, and to generate an oxygen measurement signal in dependence of the level of oxygen. The oxygen measurement signal is applied to an oxygen measurement interface unit that filters and amplifies the signal and then applies the signal to a control unit. The oxygen sensor is arranged to perform measurements inside the heart, of blood entering the left atrium of a patient's heart and is preferably arranged at, or close to, a distal end of an electrode lead connected to the medical device. The obtained oxygen measurement signal is compared, by the control unit, to a predetermined threshold level and an indication signal, indicative of the lung functionality of the patient, is generated as a comparison result. Both the obtained oxygen measurement signal as well as the indication signal are stored in a storage unit (not shown) arranged in the control unit. The stored signals are time-stamped.

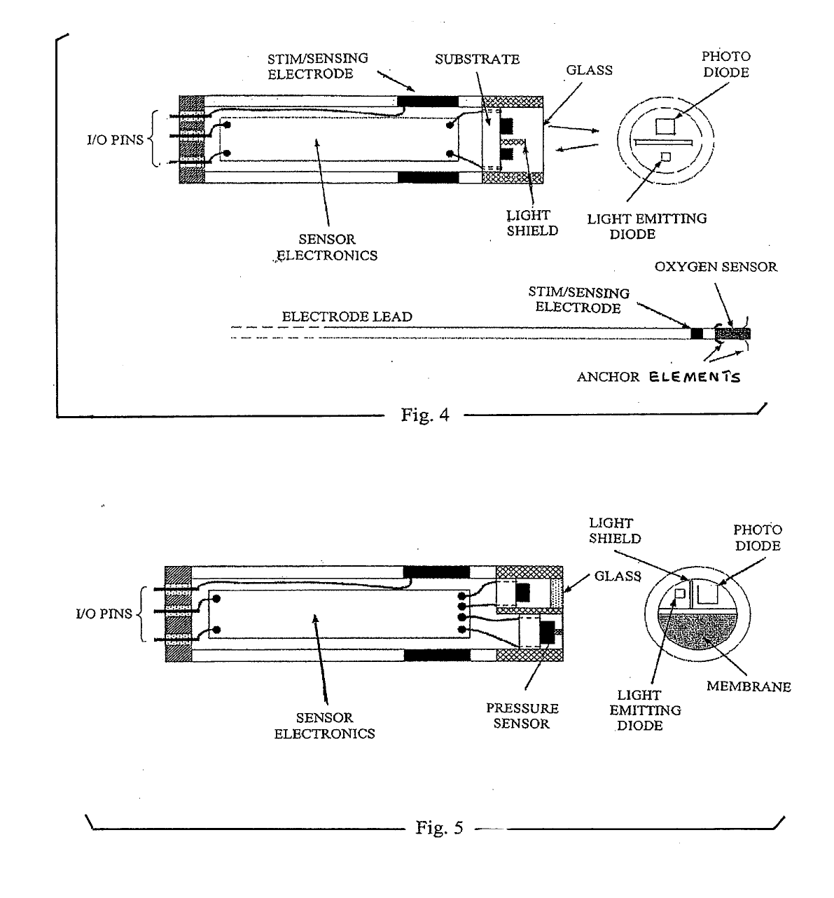

As illustrated in FIG. 4 the optical sensor preferably has a photo-diode and a light emitting diode arranged at the distal tip of the electrode lead and facing the distal direction of the lead. The photo diode and the light emitting diode are mounted at a substrate and separated by a light shield. The sensor is protected by a glass cover that is transparent for the relevant wavelengths emitted by the diode. Sensor electronics are also included in connection with the optical sensor. Input/output pins are arranged to connect the sensor electronics to electrical conductors (not shown) of the electrode lead and that in turn are connected to the oxygen measurement interface.

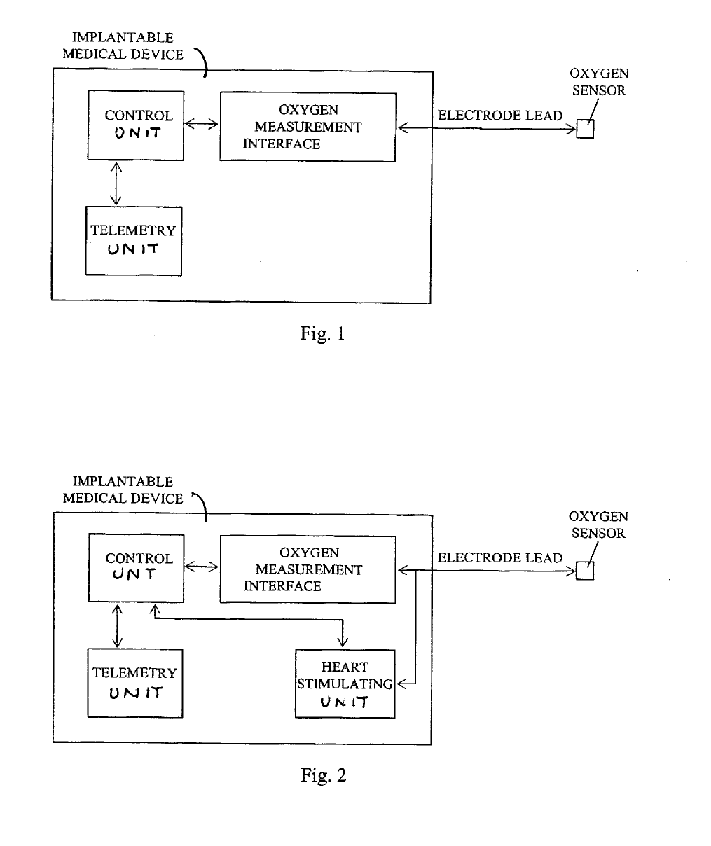

According to one embodiment of the present invention the implantable medical device is a heart stimulator which is illustrated in FIG. 2. The medical device as described with references to FIG. 1 then includes a heart stimulating pulse generator and the electrode lead includes one or many sensing and stimulating electrodes. One stimulating/sensing electrode is shown in FIG. 4. Naturally, more than one electrode lead may be connected to the medical device adapted to be arranged both in the left and right chambers of the heart. In this embodiment the oxygen measurement signal may be used to control the stimulation rate of the heart stimulator.

The electrode lead provided with the optical sensor is adapted to be placed in the left atrium of the heart and in particular to perform measurements in the left atrium from a position reached from the right atrium via the septal wall between the right and left atrium of the heart. To firmly anchor and attach the distal tip within the septal wall the distal end of the electrode lead is provided with anchor means. The anchor means are schematically illustrated in the lower part of FIG. 4 where the distal end of an electrode lead is shown. The anchor means preferably has at least two hatches separately arranged along the distal tip of the electrode lead.

As briefly mentioned above an indication signal is generated if the comparison indicates impaired lung functionality of the patient, e.g. to detect when a patient no longer may saturate the blood during exercise. In particular the indication signal is generated if the measurement signal is below a predetermined oxygen level threshold longer than a preset time period. A predetermined oxygen level threshold is 95% SaO₂.

The threshold may naturally be set to any other value, depending on the purpose of the measurement. As stated above, normal values of SaO₂ are typically 97-100% and if the value is below 92%, then the patient has severe problems.

In embodiments of the invention, the oxygen sensor is a pO₂ sensor, for example, a sensor as described in U.S. Pat. No. 5,431,172 sensitive to the levels of oxygen in arterial blood. It has been shown that the SvO₂ and SaO₂ values are correlated with the partial pressure of oxygen, pO₂, in blood and thus SaO₂ values may easily be calculated from measured pO₂ values, and vice versa.

In one embodiment of the present invention, the implantable medical device has an oxygen sensor, wherein the oxygen sensor is adapted to perform measurements inside the heart, of blood entering the left atrium of a patient's heart, to obtain a partial pressure of oxygen, pO₂, dissolved in the blood, an SaO₂ signal corresponding to the obtained pO₂ signal is calculated, and the calculated SaO₂ signal is compared to a predetermined threshold level and an indication signal is generated in dependence of this comparison, wherein said indication signal is indicative of the lung functionality of the patient.

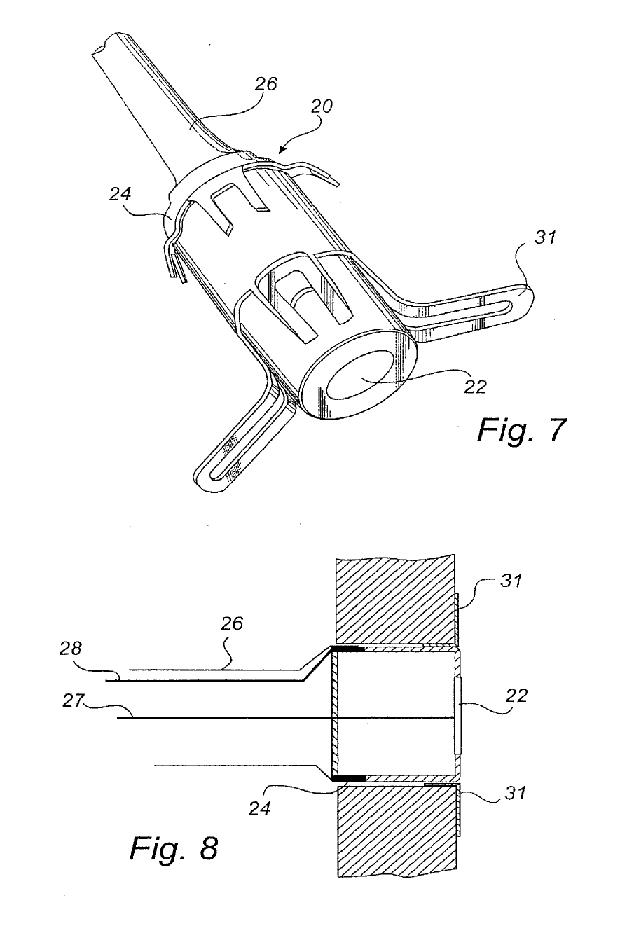

With reference now to FIGS. 7 and 8, one particular embodiment of the oxygen sensor will be discussed. The oxygen sensor 20 is arranged to penetrate septum 23 to sense the oxygen concentration of the blood in left atrium 30. The sensor comprises a working electrode 22 on which an outer surface is exposed to the blood in the left atrium 30, i.e. the sensor is located such that the outer surface of the working electrode 22 is in direct contact with the arterial blood 30. The reduction of oxygen takes place on the outer surface of the working electrode 22. According to one embodiment, the outer surface of the working electrode 22 has a coating of gold. It has been shown that the tissue overgrowth on negatively charged gold surfaces in venous blood is limited and stable over time (Holmström et. al. in Colloids and Surfaces B: Biointerfaces 11 (1998) 265-271).

Further, the sensor has a counter electrode (not shown) adapted to emit electrical current to the system and a reference electrode 24 adapted to sense an electrical potential in the blood or tissue to ensure a proper measurement potential. The sensor is connected to the implantable medical device via an implantable medical lead 26 including connections 27, 28 to the working electrode 22 and the reference electrode, respectively.

According to an embodiment, the sensor 20 is implanted transvenously and, as mentioned above, penetrates the septum 23 to place the outer surface of the working electrode 22 in contact with the blood in left atrium 30. The sensor is fastened to septum 30 by fixation means 31, for example, fixation plates or fixation elements.

Another embodiment of the present invention, a sensor for measuring the concentration of oxygen in blood by means of photoluminescence is arranged in the device for monitoring progression of heart failure in a human heart according to the present invention. Such a sensor is described in PCT/SE2007/000410 (“IMPLANTABLE DEVICES AND METHOD FOR DETERMINING A CONCENTRATION OF A SUBSTANCE AND/OR MOLECULE IN BLOOD OR TISSUE OF A PATIENT”), which is incorporated herein in its entirety by reference.

The sensor comprises a carrier in which photoluminescent molecules are embedded. The carrier is partially in contact with blood of a patient comprising oxygen for which the concentration shall be determined. The sensor further comprises a light source and a photo-detector which are optically connected to the carrier such that the light emitted from the light source can excite the photoluminescent molecules and such that the light emitted from the photoluminescent molecules in response to this excitation can be detected by the photo-detector. The carrier is selectively permeable to the oxygen molecules for which the concentration has to be determined such that oxygen molecules can diffuse from the region of the patient into the carrier. The photoluminescent molecules are selected to react with oxygen in the blood in such a manner that the characteristics of the light emitted from the photoluminescent molecules is altered because of the reaction. Depending on the concentration of oxygen molecules in the blood of the patient, the characteristics of the light emitted from the photoluminescent molecules will then be altered to different degrees. Thus, a determination of the concentration of oxygen in the blood of the patient is enabled.

In an embodiment, the light source and the photo-detector are directly arranged at a side of the carrier or in the carrier. In another embodiment, the light source and the photo-detector are optically connected to the photoluminescent molecules comprised in the carrier by means of optical fibers. The optical fibers may be arranged between the light source and the photoluminescent molecules and/or between the photo-detector and the photoluminescent molecules, which means that the light source and the photo-detector can be arranged at a certain distance from the analyzed region. The optical fibers are used to guide light from the light source and to guide light to the photo-detector, respectively.

In an embodiment, the light source and the photo-detector are fabricated on a same substrate using e.g. standard semiconductor technology. The carrier is then made of a material, such as a silicon adhesive, like e.g. the commercially available Rehau RAU-SIK SI-1511, containing the photoluminescent molecules and spread over the substrate. The substrate can then be used as a support for the light source, the photo-detector and the carrier. Such a substrate would also enable easy connection between the light source, the photo-detector and other components used to analyze the signal output of the photo-detector. The thickness of the adhesive film containing the photoluminescent molecules would typically be comprised between 0.03 and 3 mm.

In yet a further embodiment, the light source and the photo-detector can be fabricated on two separate substrates and joined together by means of the carrier, e.g. the silicon adhesive mentioned above, containing the photoluminescent molecules.

In an embodiment, a filter may be arranged in front of the photo-detector to select the range of wavelength that shall be transmitted to the photo-detector. In particular, the filter is used to eliminate the part of the excitation light emitted by the light source that is reflected by the photoluminescent molecules or by the carrier or carrier matrix.

In further embodiments, the carrier can be made as a material comprised in the group of silicone rubber (also known as polydimethylsiloxane), organosubstitute silicones such as poly(R—R′-siloxane) wherein R and R′ are one of the following {methyl, ethyl, propyl, butyl, “alkyl”, “aryl”, phenyl and “vinyl”} and not necessarily equal to each other, polyurethane, poly(1-trimethylsily-1-propyne), polystyrene, poly(methylmethacrylate), poly(vinyl chloride), poly(isobutylmethacrylate), poly(2,2,2-trifluoroethylmethacrylate), ethylcellulose, cellulose acetobutyrate, cellulose acetate, gas-permeable polytetrafluoroethylene and thermoplastic polyurethanes and copolymers, in which material the photoluminescent molecules are embedded. Further, these materials, in particular silicone rubber, thermoplastic polyurethanes and copolymers, gas-permeable polytetrafluoroethylene, are well adapted in the present invention since they are implantable biomaterials. In a particular embodiment, silicone rubber is well adapted if the sensor 1 is used to determine the concentration of oxygen in the region 10 since its permeability to oxygen is about ten times higher than in most polymeric materials, namely 6.95×10¹¹ cm⁻²s⁻¹Pa⁻¹, which corresponds to a diffusion coefficient of about 1.45×10⁻⁵ cm²s⁻¹.

The light source may be a solid state light source, preferentially a light emitting diode, which is advantageous since light emitting diodes are small and can therefore easily be incorporated in the sensor. Light emitting diodes are advantageous since the intensities, wavelengths, and time responses are controllable. Light emitting diodes can emit at various ranges, which generally extend from 390 nm to 1650 nm although not any wavelength of this range may be achieved.

The sensitivity of the photo-detector of the implantable sensor depends on the wavelength range at which the photoluminescent molecules emit. Generally, the range of wavelength at which photoluminescent molecules emit extend from 350 to 1800 nm, in particular in the range of 350-800 nm. Thus, the photo-detector shall be sensitive in this range of wavelength. As the wavelength at which selected photoluminescent molecules emit is known, the sensitivity of the photo-detector can be selected accordingly. As an example, oxyphors emit light at about 800 nm, which corresponds to near infrared. In this case, the photo-detector may be a commercially available planar InGaAs photodiode, which is sensitive to red and infrared light, i.e. in the spectral range of 800-1800 nm. Such a detector has a response time of up to 120 MHz, which is sufficiently rapid for measuring photoluminescent lifetimes and sufficiently quantitative for steady state measurements of intensities as well. The measurements of lifetimes and intensities will be described in more details later. In particular, the photo-detector may be one of the group comprised of a pn photodiode, a pin photodiode, a Schottky photodiode, an avalanche photodiode, a silicon photodiode, a planar InGaAs photodiode, a SiGe-based optoelectronic circuit and a InGaN/GaN multiple quantum well pn junction.

The photoluminescent molecules may be fluorescent molecules or phosphorescent molecules. Although it would be preferable to use fluorescent molecules as they are generally more stable over time, it is preferable to use phosphorescent molecules as phosphorescence leads to longer timescale, which then facilitates the design of the electronics in the sensor.

Since the sensor is designed to determine the concentration of oxygen in blood, it is preferable that the photoluminescent molecules are not sensitive to other gases than oxygen such as carbon monooxide which can also be found in blood. The photoluminescent molecules shall also be easily bounded to the material of the carrier, i.e. they should be compatible with the characteristics of the carrier.

Photoluminescent molecules that may be used to determined the concentration of oxygen are molecules comprised within the group of pyrene, pyrene derivatives (vinylpirene, methoxypyrene), a polyaromatic carrier, an ionic probe (ruthenium trisbypyridine) and Pd-tetra(4-carboxyphenyl)benzoporphin (Oxyphor). Additional examples are naphthalene derivatives (e.g. 2-dimethylamino-6-lauroylnaphthalene); polypyridyl complexes of transition metals, particularly Ruthenium, Osmium, or Rhenium containing fluorescent molecules {e.g. Ru(bipy)(3)(2+)(tris(2,2′-bipyridyl)ruthenium(II)chloride hexahydrate) and Ru(phen)(3)(2+)(tris(1,10-phenanthroline)ruthenium(II)chloride hydrate)}; fullerenes (C60 and C70); decacyclene; perylene and perylene derivatives (e.g.—perylene dibutylate); and metalloporphines especially Pt(II), Zn(II) and Pd(II) variants.

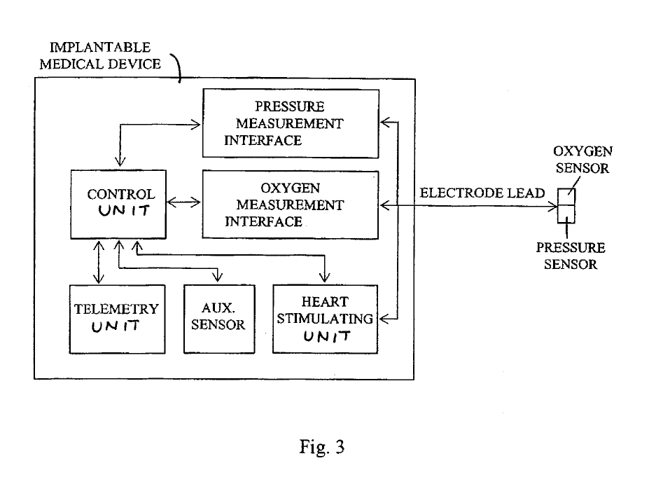

FIG. 3 illustrates another preferred embodiment of the present invention where the medical device, in addition to the oxygen sensor, further comprises a pressure sensor arranged in connection with the oxygen sensor. In the figure is also illustrated a heart stimulating pulse generator and an auxiliary sensor (to be discussed below). Both the heart stimulating pulse generator and the auxiliary sensor are optional.

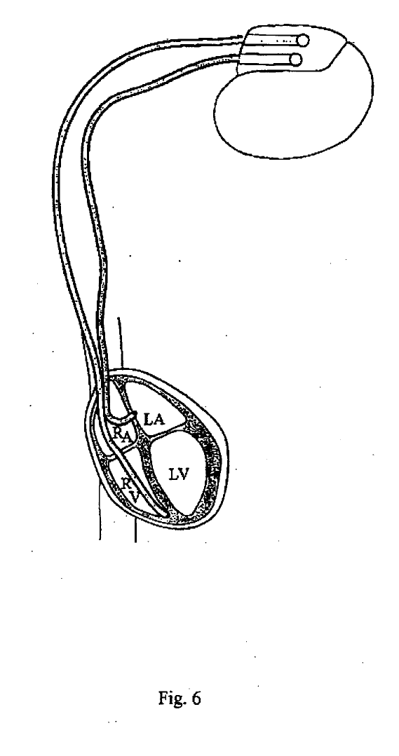

By including a pressure sensor in connection with the oxygen sensor as illustrated in FIG. 5 a very compact and versatile electrode lead is provided. As illustrated the pressure sensor is arranged at, or close to, the distal end of an electrode lead connected to the medical device.

In combination with a heart stimulating means, both the oxygen saturation signal and a pressure signal may be used to control the stimulation rate.

The medical device comprises a pressure measurement interface means connected to the pressure sensor.

Different types of pressure sensors may be used. In FIG. 5 the pressure sensor is a strain gauge pressure sensor attached to the inner side of a membrane. According to another embodiment the pressure sensor is based upon MEMS technology. In short, Micro-Electro-Mechanical Systems (MEMS) is the integration of mechanical elements, sensors, actuators, and electronics on a common silicon substrate through microfabrication technology. While the electronics are fabricated using integrated circuit (IC) process sequences (e.g., CMOS, Bipolar, or BICMOS processes), the micromechanical components are fabricated using compatible “micromachining” processes that selectively etch away parts of the silicon wafer or add new structural layers to form the mechanical and electromechanical devices.

As in the embodiment illustrated in FIG. 4 the embodiment in FIG. 5 includes input/output pins connected to electrical conductors of the electrode lead. For the other features of FIG. 5, not explicitly described here, it is referred to the description of FIG. 4.

As briefly mentioned above a further embodiment of the medical device comprises an auxiliary sensor adapted to generate an auxiliary measurement signal in dependence of a measurement influencing activity. The blood oxygen measurements are then performed when the auxiliary measurement signal fulfills specified measurement criteria. The reason to include an auxiliary sensor is to ensure that the oxygen measurements are performed with minimal disturbance or interference from other sources, e.g. when the patient is at rest or asleep.

The auxiliary sensor may be an activity sensor, a patient position sensor, or an impedance sensor. The impedance sensor preferably measures the impedance between the electrode lead and the conductive encapsulation of the implantable medical device.

The specified measurement criteria includes that the auxiliary measurement signal is below a specified level, or as an alternative, that the auxiliary measurement signal is above a specified level.

The following is an exemplary description of the function of the implantable medical device provided with an activity sensor, also functioning as a position sensor, and an impedance sensor as two auxiliary sensors.

Limit sensor values are initially set for activity and position, and limits are also set for persistence of activity and rest. The control means then starts to receive data from the position, activity and impedance sensors. Dedicated counters in the control unit are incremented as a result if detected rest and supine position, respectively, and when these counters have passed its respective limit, status flags are set to “1”. If both flags are “1” the SaO₂ measurement starts and the measurement is performed as long as the status flags indicate that the measurement conditions are acceptable.

When the oxygen measurement is performed during a predetermined time interval, e.g. 10-30 minutes, an average value is determined and compare to a threshold as discussed above. If the average value is below the threshold an impedance measurement between electrodes in relevant position in thorax may be performed in order to confirm possible pulmonary edema. An alarm may then be generated via the telemetry communication means to an external device to alert the physician.

As an alternative, the measured SaO₂ values are stored in a storage means in the control means to be further analyzed at a later follow-up procedure.

FIG. 6 illustrates an exemplary implantable medical device where the present invention is applicable. The illustrated medical device is a two-chamber heart stimulator provided with one electrode lead arranged in the right ventricle (RV) and another electrode lead, including an oxygen senor at the distal tip, arranged in the right atrium (RA), and in particular in the septal wall between the right and left atria and arranged to perform oxygen measurements in the left atrium (LV).

Although the present invention in particular relates to embodiments showing SaO₂ measured parameters, other oxygen related parameters may be used such as pO₂ as disclosed in U.S. Pat. No. 6,236,873 which is incorporated herein by reference. Measurement electrodes, adapted to perform pO₂ measurements, are then included at the distal part of the electrode lead, and the control unit then includes specific hardware and/or software to handle the measurements.

Although modifications and changes may be suggested by those skilled in the art, it is the intention of the inventors to embody within the patent warranted heron all changes and modifications as reasonably and properly come within the scope of their contribution to the art.

We claim as our invention:

1.-23. (canceled)

24. An implantable medical device comprising:
a housing configured for in vivo implantation in a living subject;
an oxygen sensor that measures a level of oxygen in oxygenated blood and that generates an oxygen measurement signal dependent on said level of oxygen;
a carrier for said oxygen sensor configured to place said oxygen sensor at a location inside the heart of the subject to cause said oxygen sensor to measure said level of oxygen in blood entering the left atrium of the heart of the subject; and
a comparator in said housing and in communication with said oxygen sensor to receive said oxygen measurement signal therefrom, said comparator comparing said oxygen measurement signal to a predetermined threshold and emitting a comparator output signal, as a result of the comparison, that is indicative of long functionality of the subject.

25. A device as claimed in claim 24 wherein said carrier is an electrode lead connected to said housing, said electrode lead having a distal end and said optical sensor being disposed approximately at said distal end of said electrode lead.

26. A device as claimed in claim 25 wherein said optical sensor comprises a photodiode and a light-emitting diode, said light-emitting diode being disposed at a distal tip of said electrode lead, facing a distal direction of said electrode lead.

27. A device as claimed in claim 24 wherein said carrier is configured to place the optical sensor in the left atrium of the heart of the subject.

28. A device as claimed in claim 27 wherein said carrier is configured to place said optical sensor in the left atrium of the heart by proceeding through the right atrium via the septal wall between the right atrium and the left atrium.

29. A device as claimed in claim 24 wherein said oxygen sensor comprises a light source that, during a measurement session, emits light at a first wavelength, and a photodetector and at least one type of photo luminescent molecules embedded in a carrier element that is selectively permeable to oxygen, at least a portion of said carrier element being configured to be in contact with arterial blood of the subject, and said light source and said photodetector being optically connected to said carrier element, said photo luminescent molecules emitting light in response to excitation by the light emitted by said light source, and said photo luminescent molecules reacting with oxygen to alter characteristics of the light emitted from the photo luminescent molecules, and said photodetector being configured to detect the light emitted from the photo luminescent molecules and to generate photodetector output signals indicative of concentration of oxygen in said arterial blood.

30. A device as claimed in claim 24 wherein said carrier comprises an electrode lead connected to said housing and having a distal end, said oxygen sensor being disposed approximately at said distal end of said electrode lead, and said electrode lead comprising a distal tip having at least one anchor element that fixes said distal tip within the septal wall of the heart of the subject.

31. A device as claimed in claim 30 wherein said electrode lead comprises two anchor elements formed by times at said distal tip of said electrode lead.

32. A device as claimed in claim 24 wherein said comparator compares said oxygen measurement signal to an oxygen level threshold, as said predetermined threshold, and comprising a processor that determines a time duration that said oxygen measurement signal is below said oxygen level threshold, said processor emitting a processor output signal indicating interstitial edema if said time duration exceeds a predetermined time duration.

33. A device as claimed in claim 32 wherein said processor employs 97% SaO₂ as said oxygen level threshold.

34. A device as claimed in claim 33 wherein said oxygen level threshold at 97% SaO₂ is a first oxygen level threshold, and wherein said processor employs a second oxygen level threshold at 92% SaO₂, and wherein said processor output is a first processor output and wherein said processor is configured to emit a second processor output if said oxygen measurement signal, indicating pulmonary edema, if said oxygen measurement signal is below said second oxygen level threshold.

35. A device as claimed in claim 24 comprising a cardiac stimulation pulse generator in said housing, and leaves connected to said stimulation pulse generator to deliver stimulation pulses in vivo to the heart of a patient at a stimulation rate, and comprising a control unit that controls the stimulation rate of the pulse generator dependent on said comparator output signal.

36. A device as claimed in claim 34 comprising a pressure sensor also carried by said carrier to place said pressure sensor in proximity to said oxygen sensor.

37. A device as claimed in claim 36 wherein said carrier comprises an electrode lead connected to said housing, said electrode lead having a distal end at which said pressure sensor and said oxygen sensor are disposed.

38. A device as claimed in claim 37 wherein said pressure sensor is a strain gauge pressure sensor.

39. A device as claimed in claim 36 wherein said pressure sensor is a MEMS technology pressure sensor.

40. A device as claimed in claim 24 comprising an auxiliary sensor that generates an auxiliary measurement signal representing a physiological parameter of the subject that is influenced by physical activity of the subject, and comprising a control unit that causes measurement of said level of oxygen by said oxygen sensor only when said auxiliary measurement signal satisfies predetermined measurement criteria.

41. A device as claimed in claim 40 wherein said auxiliary sensor is an accelerometer sensor;

42. A device as claimed in claim 40 wherein said auxiliary sensor measures subject posture.

43. A device as claimed in claim 40 wherein said control unit enables measurement of said level of oxygen by said oxygen sensor when said auxiliary measurement signal is below a predetermined level, as said specified measurement criteria.

44. A device as claimed in claim 40 wherein said control unit enables measurement of said level of oxygen by said oxygen sensor when said auxiliary measurement signal is above a predetermined level, as said specified measurement criteria.

45. An implantable medical device comprising:
a housing configured for in vivo implantation in a living subject;
an oxygen sensor that measures a partial pressure of oxygen dissolved in blood and that emits a small pO₂ signal dependent on the measured partial pressure of oxygen;
a carrier for said oxygen sensor configured to place said oxygen sensor at a location inside the heart of the subject to cause said oxygen sensor to measure said partial pressure of oxygen in blood entering the left atrium of the heart of the subject;
a processor in said housing and in communication with said oxygen sensor, that calculates an SaO₂ signal corresponding to said small pO₂ signal; and
a comparator that compares said SaO₂ signal to a predetermined threshold level and that emits a comparator output, dependent on said comparison, indicating lung functionality of the subject.
